(19) United States
(12) Patent Application Publication
(10) Pub. No.: US 2020/0016055 A1
(43) Pub. Date: Jan. 16, 2020

(54) LIQUOR-BASED UNDERARM DEODORANT

(71) Applicants: Erica Feucht, Livermore, CO (US); Jason Feucht, Livermore, CO (US)

(72) Inventors: Erica Feucht, Livermore, CO (US); Jason Feucht, Livermore, CO (US)

(21) Appl. No.: 16/578,342

(22) Filed: Sep. 22, 2019

Related U.S. Application Data
(63) Continuation-in-part of application No. 15/848,981, filed on Dec. 20, 2017.

(51) Int. Cl. A61K 8/34; A61K 8/02; A61K 8/04; A61K 8/96; A61K 8/92; A61K 8/73; A61K 8/9789; A61Q 15/00; A61K 8/11

(52) U.S. Cl. CPC A61K 8/34; A61K 8/0229; A61K 8/0208; A61K 8/046; A61K 8/11; A61K 8/92; A61K 8/737; A61K 8/9789; A61Q 15/00; A61K 8/96

(57) ABSTRACT

An underarm deodorant has a solvent and a potable alcohol component having a distinctive fragrance, taste, or both, and a denaturing agent. The underarm deodorant can also include an antiperspirant component and a chemical pheromone. The antiseptic and alcohol components can be encapsulated in time-release polymer enclosures. The alcohol component can be in the form of beer, wine, whiskey, vodka, scotch, rye, gin, rum, bourbon, tequila, and other liquors. A method of producing the underarm deodorant is also disclosed.

### RELATED PATENT APPLICATION

The present application is a continuation-in-part of co-pending patent application titled LIQUOR-BASED UNDERARM DEODORANT, application Ser. No. 15/848,981, filed Dec. 20, 2017, now abandoned, and hereby incorporates the teachings therein.

### FIELD OF THE INVENTION

This invention relates to deodorants and, more particularly, to a potable, liquor-based underarm deodorant.

### BACKGROUND OF THE INVENTION

Human skin is naturally populated with numerous micro-organisms. These organisms are nourished by various skin secreted substances, skin cell debris, breakdown products of the skin and the organisms themselves. The “skin secretions” are eccrine and apocrine sweat, and lipid-soluble sebum. Eccrine sweat is normally odorless and remains odorless after secretion, although odoriferous food and drug substances may be excreted with it. Apocrine glands are normally associated with hair follicles and are confined mainly to the groin, perianal, areola and armpits. They produce a scanty, milky substance that is odorless upon secretion, but becomes odoriferous upon bacterial decomposition. Apocrine glands are considered to be a primary contributor for malodor. The sebaceous glands are distributed over the skin surface except the palms and dorsae. They are most numerous on the scalp, forehead, face, back, and chest. The secretion, sebum, consists mainly of fatty materials, wax esters, cholesterol and its esters, and squalene.

Body odor is most commonly caused by fatty acids on skin and from malodors from bacterial sources. The unpleasant odors are mainly organic molecules which have different structures and functional groups, such as amines, acids, alcohols, aldehydes, ketones, phenolics, polycyclics, indoles, aromatics, polyaromatics, etc. They can also be made up of sulfur-containing functional groups, such as, thiol, mercaptan, sulfide and/or disulfide groups.

Odor causing bacteria and fungi often flourish in warm, moist conditions, particularly where they have easy access to nourishment such as skin secretions and skin cell debris. Accordingly, attempts have been made to control odor through moisture absorption, depriving the bacteria responsible for body odor of the moist/humid environment they need to proliferate and grow. Such efforts include the use of powders and/or antiperspirants. Powders and powder-based compositions may be difficult to apply and have limited absorption capabilities. Therefore, use of body powders is generally undesirable and/or ineffective for day to day body odor control for the entire body. Antiperspirants are not useful in a body odor control product for use over the entire body as they may interfere with the body's thermal regulatory process by inhibiting perspiration through the action of astringent salts. Additionally, such salts may be irritating to a large number of users, particularly when applying them to sensitive areas such as the pelvic region.

Known deodorants attempt to control odor through a variety of means. Deodorants may include antibacterial compounds which help destroy and/or control the amount of bacteria present on the skin, thereby minimizing odor produced via bacterial metabolism of the skin secretions.

Some 100 years ago, deodorants and antiperspirants were invented to mask human body odor. The first patents for deodorants were filed in the 1860s, when inventors used sanitizing agents such as ammonium chloride, sodium bicarbonate, and formaldehyde to kill microorganisms. Zinc oxide, still commonly found in deodorants, was one of the first ingredients in the early deodorants. Current deodorant ingredients vary vastly, from antibiotics to citronella oil. All are intended to control bacterial growth.

Prior to those inventions, people washed themselves and/or used fragrances to accomplish this odor masking. Ancient Egyptians had perfumes specifically intended to cover human body odor. But formulas to block odor have only been in wide use for only a century.

Over the years, many perfumes have been used to mask body odors. Not only are such perfumes often inadequate at fully concealing the body odors, very often they are irritating to the user's skin. Additionally, the perfume odor itself may be irritating or offensive to the user's respiratory system and/or olfactory senses, as well as to nearby individuals.

Deodorants and antiperspirants differ from perfumes because they not only mask human body odors, they thwart its production. Deodorants typically include antiseptic ingredients that kill or disable bacteria living in the armpit. That region's warm and moist environment is an optimal habitat for corynebacteria and other sweat-eating microorganisms. These life-forms generate malodorous waste by-products such as trans-3-methyl-2-hexenoic acid, the compound responsible for the characteristic odor of human sweat.

Whereas deodorants attempt to kill the odor-producing microorganisms, antiperspirants block sweat pores, preventing the flow of nutrients to armpit microorganisms. Both products typically include a scent, usually in the form of perfume. Deodorants work by neutralizing the smell of the sweat and by antiseptic action against bacteria. Deodorants do not interfere with sweating, a natural cooling process, as do antiperspirants.

The array of active ingredients in deodorant contrasts with those found in antiperspirants. Antiperspirants have always relied on aluminum salts, such as aluminum chloride, to stop the flow of sweat. More recently, antiperspirant manufacturers have used aluminum formate, aluminum chlorohydroxide, and aluminum zirconium tetrachlorohydrate.

Aside from active ingredients, the way that deodorants and antiperspirants are applied to the armpit has evolved since their inception. The same holds true for the solvents in which the active ingredients are dissolved or suspended. Early antiperspirants suspended their active ingredient in alcohol or, more often, acids, and had to be swabbed with cotton balls on the armpit. These formulations were slow to dry and prone to irritating skin and ruining clothing. Likewise, early deodorants were often sold as creams, which were rubbed by hand on the armpit, often leaving these zones and clothing greasy. The roll-on deodorant was an application mechanism invented in the 1940s. Sticks, gels, and aerosols emerged in the decades thereafter.

Although these relatively new deodorant and antiperspirant products and application or delivery mechanisms improved the aesthetics and ease of application, the biggest nonactive ingredient innovation came in the 1970s, when quick-dry cyclomethicones, also known as methylsiloxanes, were used. Cyclomethicone solvents are now widespread in many antisweat products because they do not irritate skin and they evaporate quickly, leaving behind the deodorant or antiperspirant's active ingredient, but no greasiness or stickiness.

Deodorant compositions have been described in the chemical and cosmetic literature, generally in the form of emulsion sticks, but also may be aerosols, roll-ons, pads, pump sprays, and even soap bars. In recent decades, makers of deodorants and antiperspirants began to encapsulate active ingredients in time-release polymer enclosures so the products last longer.

Common chemicals in deodorants are purified water, guar gum, vegetable glycerin, glycerol stearate, patchouli, sandalwood essential oil, and lavender. Purified water is an active absorber, which, when it comes into contact with air, absorbs carbon dioxide, making it acidic. Guar is digestive fiber used in the food industry as a thickening agent. It helps control the rate of the process absorbing of nutrients, including sugar, spreading it over a longer period of time. Vegetable glycerin can be added to increase moisturizing qualities. Glycerol stearate is a smooth, white powder with odor and taste. Patchouli is fresh oil used in skin care when cracked, dry, itchy, or mature. It is strengthening oil often used as a fixative for perfume blends. Sandalwood essential oil is used for perfumery, cosmetics, and aromatherapy. Lavender is moisturizing to skin.

Coupled with the goal of masking or eliminating body odor, modern perfumes, scents, body washes, after-shave lotions, balms, and gels have been produced to increase sexual magnetism between the wearer of such products and those they wish to influence or entice. It has been found that both the scent and taste of certain alcohol beverages can act as an intoxicant or an aphrodisiac. Moreover, pheromones have been used in various products with similar results. Along these lines, the growing field of aromatherapy is based on using natural substances to enhance psychological and physical well-being.

It is with this realization that the present inventors have formulated deodorants having alcohol fragrances and tastes. These formulations can have the effect of heightening erotic stimulation for parties in intimate situations.

### DESCRIPTION OF RELATED ART

U.S. Pat. No. 9,511,034 issued to Garrett for METHOD FOR APPLYING A SKIN TREATMENT issued on Dec. 6, 2016, describes a skin treatment for managing skin conditions ranging from scars, including keloids, to burns and wrinkles. The skin treatment may comprise several components including a cleansing solution, a silicone elastomer, and a mineral powder that may be utilized to treat and camouflage the affected skin. In one embodiment, a cleansing component is used to prime a skin area, cleansing and treating the area so that a silicone component can heal the skin.

U.S. Pat. No. 9,789,038 issued to Fan, et al. for ANTIPERSPIRANT/DEODORANT COMPOSITIONS issued on Oct. 17, 2017, describes a composition comprising at least one active chosen from at least one antiperspirant active and at least one deodorant active; a first gellant chosen from at least one fatty alcohol and at least one hydrocarbon of the formula CₙH₂ₙ₊₂, wherein n is about 20 to about 100, and the hydrocarbon is at least 90% linear; at least one soybean oil having an iodine value of greater than 0 to about 20; and at least one silicone.

U.S. Pat. No. 4,781,917 issued to Luebbe, et al. for ANTIPERSPIRANT GEL STICK issued on Nov. 1, 1988, describes antiperspirant gel stick compositions substantially free of unbound water comprising from about 5 to about 50% of a solubilized antiperspirant active, from about 7% to about 35% of intermediate polarity emollients, from about 1% to about 5% of a benzylidene sorbitol, from about 15% to about 75% of a polar solvent, from about 1% to about 20% of a coupling agent and from about 0.5% to about 10% of a buffering agent. Also described is a method for treating or preventing perspiration and malodor associated with human underarm perspiration.

Japanese Patent Application No. JP2002/080335 on application by Sakiguchi, describes a cool feeling-imparting deodorant cosmetic high in safety and capable of affording sufficient deodorant effect. The deodorant cosmetic is characterized by containing 0.1-5 mass % of a cool-feel sustaining ingredient, 0.01-2 mass % of a cool feeling-imparting substance, and 0.01-40 mass % of one or more antiperspirant, germicide, and deodorizer, the cooling sensation maintaining component being a menthol derivative, L-methyl glyceryl ether, L-methyl lactate, or a cooling-agent-containing powder, the cooling agent being menthol, camphor, or eucalyptus oil, and ethanol used as a solvent and/or an antiseptic.

U.S. Pat. No. 6,183,730 issued to Guskey, et al. for Antiperspirant and deodorant compositions containing cyclohexasiloxane issued on Feb. 6, 2001, describes antiperspirant and deodorant compositions comprising select concentrations of cyclohexasiloxane as a volatile silicone material from about 0.1% to about 50% by weight of an antiperspirant active, and from about 0.1% to about 50% by weight of a suspending agent, wherein the compositions are preferably substantially free of cyclotetrasiloxane.

U.S. Pat. No. 5,272,134 issued to Berliner for FRAGRANCE COMPOSITIONS AND OTHER COMPOSITIONS WHICH CONTAIN HUMAN PHEROMONES issued on Dec. 21, 1993, describes non-therapeutic fragrance compositions and other compositions containing an odorant and a naturally occurring human pheromone. The invention also concerns fragrance compositions containing mixtures of naturally occurring human pheromones. The human pheromones disclosed are steroids which desirably belong to two distinct chemical classes: 16-Androstenes and Estrenes.

U.S. Pat. No. 4,818,522 issued to Ferentchak, et al. for ENCAPSULATION OF ADJUVANTS WITHIN ANTIPERSPIRANT ACTIVES issued on Apr. 4, 1989, describes antiperspirant formulations in which water-immiscible adjuvants are encapsulated in thick-walled, hollow, substantially spherical particles of antiperspirant active, which is preferably a water-soluble compound or complex of a polyvalent metal or metals. The adjuvant may be a fragrance, an antibacterial, antimicrobial or antifungal agent, a deodorant, or other dermatological preparation.

U.S. Pat. No. 4,634,588 issued to Moroe for DEODORANT issued on Jan. 6, 1987, describes what is commonly referred to in the United States as a mouthwash, but inaccurately referred to as a “deodorant” in the patent, which mouthwash is derived from brewed wine or residual fermentation materials produced in the manufacture of brewed wine. In one aspect, the mouthwash is manufactured from the brewed wine by evaporating water and alcohols therefrom. The residual fermentation materials are first extracted with ethanol to obtain an ethanol-extracted liquid.

United States Published Patent Application No. 2008/0207871 on an application filed by Seiler for PREPARATIONS CONTAINING HYPERBRACHED POLYMERS issued on Aug. 28, 2008, describes preparations comprising at least one low molecular weight substance and at least one hyperbranched polymer, wherein the hyperbranched polymer comprises a hydrophilic core having polyester units and hydrophobic end groups, said hyperbranched polymer having a molecular weight greater than or equal to 6000 g/mol and a hydroxyl number in the range from 0 to 200 mg KOH/g, the degree of branching being in the range from 20 to 70%, and said hyperbranched polymer having a melting point of at least 30° C.

U.S. Pat. No. 4,224,307 issued to Thiele, et al. for MOUTHWASH AND METHODS issued on Sep. 23, 1980, describes a liquefied mouthwash composition of an effective amount of a non-necrotic fatty acid compound prepared from an unsubstituted, unsaturated fatty acid having at least one double bond, water, an effective amount of a buffering agent, 1 to 10 percent of ethanol, and at least one oil soluble flavorant. The pH of the liquefied composition is between 9 and 11.

### SUMMARY OF THE INVENTION

In accordance with the present invention, there is provided an underarm deodorant that has a solvent, an alcohol component having a distinctive fragrance, taste, or both, and a denaturing agent. The underarm deodorant can also include an antiperspirant component and a chemical pheromone. The antiseptic and alcohol components can be encapsulated in time-release polymer enclosures. The alcohol component can be in the form of beer, whiskey, vodka, scotch, rye, gin, rum, bourbon, and other liquors. A method of producing the underarm deodorant is also disclosed.

It is therefore an object of the invention to provide a deodorant having a scent and/or taste of an alcohol beverage.

It is a further object of the present invention to provide an antiperspirant having a scent and/or taste of an alcohol beverage.

It is a further object of the present invention to provide one or both of the aforementioned products additionally having a scent and/or taste of cannabis.

It is a further object of the present invention to provide one or both of the aforementioned products additionally having a pheromone component.

These and other objects and advantages of the present invention are more readily apparent with reference to the following detailed description and the accompanying drawing.

### BRIEF DESCRIPTION OF THE DRAWINGS

A complete understanding of the present invention may be obtained by reference to the accompanying drawing, when considered in conjunction with the subsequent detailed description, in which:

FIG. 1 is a high level flow chart of the steps used to produce the liquor-based deodorant in accordance with the present invention.

### DETAILED DESCRIPTION OF THE PREFERRED EMBODIMENT

Although the following detailed description contains specific details for the purposes of illustration, those of ordinary skill in the art will appreciate that variations and alterations to the following details are within the scope of the invention. Accordingly, the exemplary embodiments of the invention described below are set forth without any loss of generality to, and without imposing limitations upon, the claimed invention.

The underarm deodorant of the invention has a solvent and an alcohol component having a distinctive fragrance, taste, or both. The alcohol component can be liquor in a form including but not limited to beer, wine, whiskey, vodka, scotch, rye, gin, rum, bourbon, tequila, other liquors, or even mixed drinks.

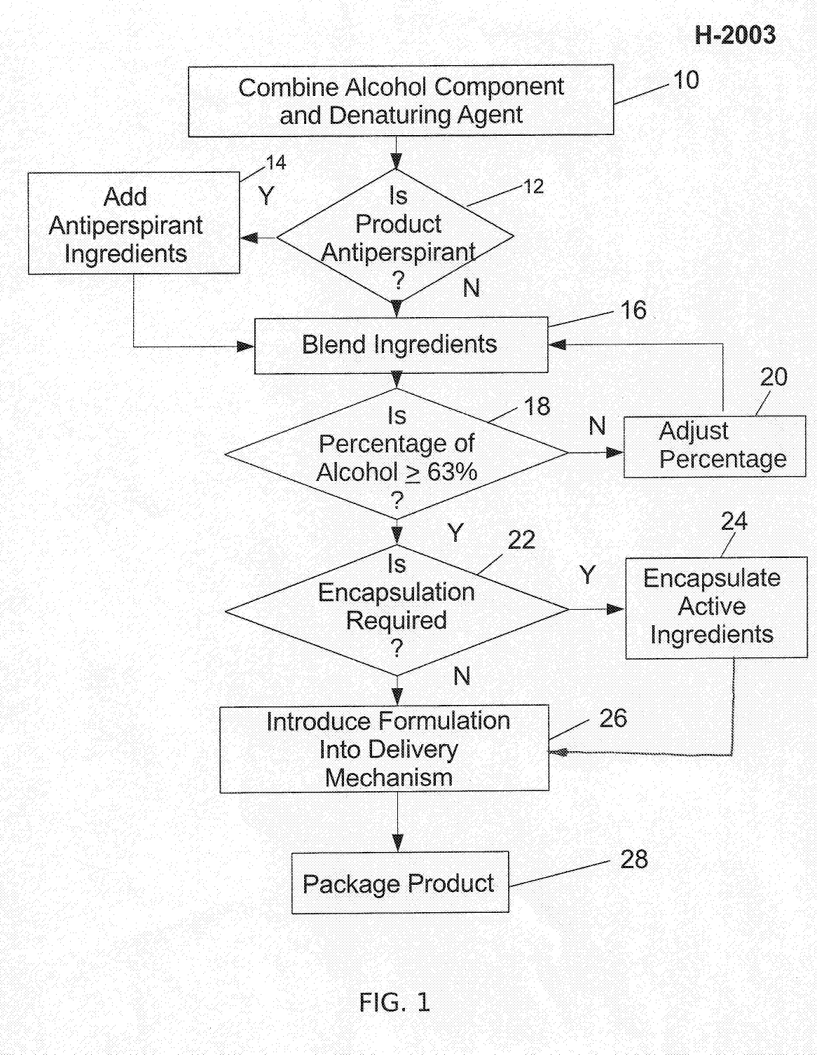

Referring now to FIG. 1, there is shown a flow chart of the steps used to produce the liquor-based deodorant in accordance with the present invention. The first step in producing the deodorant is combining a solvent and a potable alcohol or scent thereof, such as beer, wine, whiskey, vodka, scotch, rye, gin, rum, bourbon, or tequila with a denaturing agent, step 10. Other liquors or mixed drinks may also be used and are considered to be within the scope of the present invention and are preferable to industrial produced alcohol do to purity and toxicity. Moreover, other substances may be combined with the alcohol, including but not limited to cannabinoids and one or more chemical pheromones.

The denaturing agent is used to render the underarm deodorant unpalatable. The denaturing agent comprises salt, essential oils, bitter herbs, and/or other substances known in the art that have the effect of making the underarm deodorant unpalatable.

The solvent can include an antibacterial compound, purified water, guar gum, vegetable glycerin, glycerol stearate, patchouli, sandalwood essential oil, and/or lavender. In the preferred embodiment, the solvent comprises a cyclomethicone. Moreover, salt, arrowroot, and antibacterial tea can be included in the solvent to extend the effectiveness of the formulation and inhibit growth of bacteria. In the preferred embodiment, the antibacterial tea used is Umckaloabo, Greek Mountain Tea, or Gotu Kola.

Optionally, step 12, at least one antiperspirant component that preferably includes an aluminum salt may be combined with the aforementioned ingredients, step 14. The alcohol component, the denaturing agent, and, optionally, the antiperspirant ingredients are the blended, step 16, to ensure all of the ingredients are consistently mixed throughout the formulation. After blending, step 16, the formulation is tested, step 18, to ensure the alcohol component is at least 63% by weight of the formulation. If the alcohol component is less than 63% by weight of the formulation, step 18, more alcohol composition is added thereto, step 20.

If encapsulation of active ingredients is required, step 22, such active ingredients are, in fact, encapsulated in time-release polymer enclosures, step 24, in a manner well known in the art.

The formulation is then formed into a delivery mechanism, step 26, such as one including but not limited to a stick, gel, aerosol, pad, pump spray, or soap bar. Other delivery mechanisms may also be devised without exceeding the scope of the present invention.

The delivery mechanism is then packaged in a convenient form for consumers, step 28, that includes space for identifying the product with an appropriate trademark, list of ingredients, warnings, and method of application.

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims.

What is claimed is:

1. An underarm deodorant produced by a formulation, comprising:
a) a solvent;
b) an alcohol component being at least 63% by weight of the underarm deodorant formulation having a distinctive fragrance; and
c) a denaturing agent for rendering the underarm deodorant unpalatable, the denaturing agent comprising salt.

2. The underarm deodorant in accordance with claim 1, further comprising:
d) an antiperspirant component.

3. The underarm deodorant in accordance with claim 1, further comprising:
d) a chemical pheromone.

4. The underarm deodorant in accordance with claim 1, wherein the alcohol component (b) is encapsulated in time-release polymer enclosures.

5. The underarm deodorant in accordance with claim 2, wherein at least one of the alcohol and antiperspirant components (b), (d) is encapsulated in time-release polymer enclosures.

6. The underarm deodorant in accordance with claim 1, wherein the underarm deodorant delivery mechanism is in a form selected from one of a group of application mechanisms consisting of:
i) emulsion sticks,
ii) aerosols,
iii) roll-ons,
iv) pads,
v) pump sprays, and
vi) soap bars.

7. The underarm deodorant in accordance with claim 1, wherein the alcohol component comprises at least one chosen from a group consisting of:
i) beer,
ii) wine,
iii) whiskey,
iv) vodka,
v) scotch,
vi) rye,
vii) gin,
viii) rum,
ix) bourbon, and
x) tequila.

8. The underarm deodorant in accordance with claim 2, wherein the antiperspirant comprises an aluminum salt and the solvent comprises a cyclomethicone.

9. The underarm deodorant in accordance with claim 2, further comprising:
e) a cannabis-scented component.

10. The underarm deodorant in accordance with claim 3, further comprising:
e) a cannabis-scented component.

11. The underarm deodorant in accordance with claim 1, wherein the denaturing agent further comprises art least one of a group consisting of:
i) bitter herbs; and
ii) essential oils.

12. The underarm deodorant in accordance with claim 1, wherein the solvent comprises at least one substance chosen from a group consisting of:
i) an antibacterial compound;
ii) purified water;
iii) guar gum;
iv) vegetable glycerin;
v) glycerol stearate;
vi) patchouli; and
vii) sandalwood essential oil.

13. The underarm deodorant in accordance with claim 1, further comprising:
d) antibacterial tea to extend the effectiveness of the formulation and inhibit growth of bacteria.

14. A process of producing an underarm deodorant by producing a formulation, the steps comprising:
a) combining an alcohol component and a denaturing agent, the denaturing agent comprising salt for rendering the underarm deodorant unpalatable;
b) adding an antiperspirant if the underarm deodorant is also to function as an antiperspirant;
c) blending the alcohol component, the denaturing agent and, optionally, the antiperspirant;
d) adjusting the quantity of alcohol component if the amount of alcohol is less than 63% by weight of the formulation; and
e) introducing the formulation into a delivery mechanism.

15. The process of producing an underarm deodorant in accordance with claim 14, further comprising:
f) encapsulating the alcohol component, the denaturing agent and, optionally, the antiperspirant.

16. The process of producing an underarm deodorant in accordance with claim 14, wherein the underarm deodorant delivery mechanism is in a form selected from one of a group of application mechanisms consisting of:
i) emulsion sticks,
ii) aerosols,
iii) roll-ons,
iv) pads,
v) pump sprays, and
vi) soap bars.

17. The process of producing an underarm deodorant in accordance with claim 14, wherein the alcohol component comprises at least one chosen from a group consisting of:
i) beer,
ii) wine,
iii) whiskey,
iv) vodka,
v) scotch,
vi) rye,
vii) gin,
viii) rum,
ix) bourbon, and
x) tequila.

18. The process of producing an underarm deodorant in accordance with claim 14, wherein the antiperspirant comprises an aluminum salt and the solvent comprises a cyclomethicone.

19. The process of producing an underarm deodorant in accordance with claim 14, wherein the denaturing agent further comprises art least one of a group consisting of:
i) bitter herbs; and
ii) essential oils.

20. The process of producing an underarm deodorant in accordance with claim 14, wherein the solvent comprises at least one substance chosen from a group consisting of:
i) an antibacterial compound;
ii) purified water;
iii) guar gum;
iv) vegetable glycerin;
v) glycerol stearate;
vi) patchouli; and
vii) sandalwood essential oil.
